(19) United States
(12) Patent Application Publication
(10) Pub. No.: US 2019/0218256 A1
(43) Pub. Date: Jul. 18, 2019

(54) MUTANT LIGHT-INDUCIBLE ION CHANNEL OF CHANNELRHODOPSIN

(71) Applicant: Max-Planck-Gesellschaft zur Förderung der Wissenschaften e.V., Muenchen (DE)

(72) Inventors: Ernst Bamberg, Kelkheim (DE); Phil Wood, Schmelz/Saar (DE); Thomas Mager, Frankfurt (DE)

(21) Appl. No.: 16/306,673

(22) PCT Filed: Jun. 2, 2017
(86) PCT No.: PCT/EP2017/063425

§ 371 Date: Dec. 3, 2018

(30) Foreign Application Priority Data
Jun. 3, 2016 (EP) 16172992.6

(51) Int. Cl. C07K 14/405; C12N 15/86; C07K 14/705; C12N 7/00; A61K 9/00; A61P 27/02

(52) U.S. Cl. CPC C07K 14/405; C12N 15/86; A61P 27/02; C12N 7/00; A61K 9/0019; C07K 14/705

(57) ABSTRACT

The invention relates to mutant light-inducible ion channel having improved properties as compared to the parent channel, nucleic acid constructs encoding same, expression vectors carrying the nucleic acid construct, cells comprising said nucleic acid construct or expression vector, and their respective uses, as well as non-human animals comprising the mutant light-inducible ion channel, the nucleic acid construct or the expression vector as disclosed herein.

The invention relates to mutant light-inducible ion channel having improved properties, nucleic acid constructs encoding same, expression vectors carrying the nucleic acid construct, cells comprising said nucleic acid construct or expression vector, and their respective uses, as defined in the claims.

### BACKGROUND OF THE INVENTION

The light-gated, inwardly rectifying cation channels, channelrhodopsin-1 (ChR1) and channelrhodopsin 2 (ChR2) has become a preferred tool for the targeted light-activation of neurons both in vitro and vivo¹⁻⁵. Although wild-type (WT) ChR2 can be employed for light-induced depolarization, there is an ongoing search for ChR2 mutants with faster kinetics and increased light-sensitivity for potential future clinical applications (WO 03/084994 and⁶⁻⁸).

Since the first description in 2002 and 2003 a set of different variants of ChR2 including a red-light absorbing channelrhodopsin are described. For different purposes ChR's were modified with respect to the kinetics, ion selectivity as well as light absorption. Examples are the red light absorbing channelrhodopsin Chrimson (WO 2013/071231 and⁹), the Volvox channelrhodopsin (VChR1)¹⁰ and the chimera ReaChR (ChR2CV1, CV2, Red absorbing ChannelRhodopsin; U.S. Pat. No. 8,759,492 B2, and¹¹). Also described in the art is the Ca²⁺ permeable mutant L132C of channelrhodopsin 2 (CatCh; WO 2012/032103, and¹²). Previous research has demonstrated that mutations at positions C128 and D156 in helix 3 and 4, respectively, resulted in markedly slowed channel kinetics with open life-times up to 30 minutes and more, yielding a 500-fold or even higher light-sensitivity^{6,7}. These C128 and D156 mutants can be switched off at variable open times by red light. Despite the superior light-sensitivity, their slow closing kinetics remains a limiting factor for their applicability.

The ChR2 kinetics are a major issue, because the light sensitivity is regulated via the open time of the channel. This is due to the invariance of other channel parameters like single channel conductance, open probability, quantum efficiency. In other words, channels with a long open time reach the maximal activity at low light intensity, whereas short living channels need more light to reach saturation with respect to light saturation. Although ‘fast’ channels need more light for the activation, high speed is indispensable for many applications in neurobiology because of the high frequency firing rate of different neuronal cells. This is valid e.g. for ganglion cells in the auditory system for interneurons in the brain, which reach firing rates up to 1000 Hz.

Accordingly, there is still a need for mutant light-inducible ion channels exhibiting faster response kinetics.

### SUMMARY OF THE INVENTION

The inventors performed a systematic study on different channelrhodopsins by modification of position F219 in helix 6 of the seven transmembrane helix motif¹⁶. It could be demonstrated that mutation of position F219 in helix 6 in WT ChR2 accelerates the closing time (off-kinetics) of the channel. Also the Ca²⁺ permeable mutant L132C (CatCh) is accelerated by the modification in helix6, and an increase of Ca²⁺ permeability was observed. The newly discovered effect could be demonstrated in ChR2, the Volvox channelrhodopsin (VChR1; accession number EU622855), and in the chimera ReaChR (ChR2CV1, CV2, Red absorbing Channelrhodopsin; accession number KF448069). It is important to note that the considered positions are homologous in the different channelrhodopsins in helix 6. An overview of the modifications in helix 6 is given in FIG. 1.

The present disclosure describes a general way to modify channelrhodopsins with respect to speed by combination of the properties of the different Channelrhodopsins. The use of these new variants will provide a light stimulation of neurons up to their limits of 800 to 1000 Hz.

The modifications yield also an increase of the Ca²⁺ permeability, which is supposed to be used in the plasma membrane and in the membrane of intracellular Ca stores, the sarcoplasmic reticulum, the endoplasmic reticulum or mitochondria as a useful tool to influence the Ca²⁺ concentration in the cytosol.

An experimental verification for the increased speed and enhanced Ca²⁺ permeability was tested in NG108-15 cells (neuroblastoma cells), in HEK293 cells an hippocampal cells from the mouse brain.

Since it is known that a cell's inner membrane surface potential is strongly influenced by Ca²⁺, modifying submembraneous intracellular Ca²⁺ levels will lead to depolarization of the membrane and in neurons to activation of voltage-gated Na⁺ channels. The relatively high light-gated Ca²⁺-influx elevates the inner membrane surface potential and activates Ca²⁺-activated large conductance potassium (BK) channels. An increase in [Ca²⁺]ᵢ elevates the internal surface potential, facilitating activation of voltage-gated Na⁺-channels and indirectly increasing light-sensitivity. Repolarization following light-stimulation is markedly accelerated by Ca²⁺-dependent BK-channel activation.

Accordingly, disclosed is a mutant light-inducible ion channel, wherein the mutant light-inducible ion channel comprises an amino acid sequence which has at least 66% similarity/homology to the full length sequence of SEQ ID NO: 1 (ChR-2), and wherein the mutant light-inducible ion channel only differs from its parent light-inducible ion channel by a substitution at a position corresponding to F219 in SEQ ID NO: 1,

which substitution increases the off-kinetics of the mutant channel as compared to the parent channel, when compared by patch-clamp measurements in the whole cell configuration at a clamp potential of −60 mV, a bath solution of 140 mM NaCl, 2 mM CaCl₂, 2 MgCl₂, 10 mM HEPES, pH 7.4, and a pipette solution of 110 mM NaCl, 2 mM MgCl₂, 10 mM EGTA, 10 mM HEPES, pH 7.4.

Further disclosed is a mutant light-inducible ion channel, wherein the mutant light-inducible ion channel comprises an amino acid sequence which has at least 66% similarity/homology to the full length sequence of SEQ ID NO: 2 (VChR1), and wherein the mutant light-inducible ion channel only differs from its parent light-inducible ion channel by a substitution at a position corresponding to F214 in SEQ ID NO: 2,

which substitution increases the off-kinetics of the mutant channel as compared to the parent channel, when compared by patch-clamp measurements in the whole cell configuration at a clamp potential of −60 mV, a bath solution of 140 mM NaCl, 2 mM CaCl₂, 2 MgCl₂, 10 mM HEPES, pH 7.4, and a pipette solution of 110 mM NaCl, 2 mM MgCl₂, 10 mM EGTA, 10 mM HEPES, pH 7.4.

Also disclosed is a mutant light-inducible ion channel, wherein the mutant light-inducible ion channel comprises an amino acid sequence which has at least 66% similarity/homology to the full length sequence of SEQ ID NO: 3 (ReaChR), and wherein the mutant light-inducible ion channel only differs from its parent light-inducible ion channel by a substitution at a position corresponding to F259 in SEQ ID NO: 1,

which substitution increases the off-kinetics of the mutant channel as compared to the parent channel, when compared by patch-clamp measurements in the whole cell configuration at a clamp potential of −60 mV, a bath solution of 140 mM NaCl, 2 mM CaCl₂, 2 MgCl₂, 10 mM HEPES, pH 7.4, and a pipette solution of 110 mM NaCl, 2 mM MgCl₂, 10 mM EGTA, 10 mM HEPES, pH 7.4.

Further provided is a nucleic acid construct, comprising a nucleotide sequence coding for the light-inducible ion channel as disclosed herein.

Also provided is an expression vector, comprising a nucleotide sequence coding for the light-inducible ion channel or the nucleic acid construct as disclosed herein. Moreover, a cell is provided, comprising the nucleic acid construct or the expression vector as disclosed herein.

In still another aspect, the invention provides the use of the light-inducible ion-channel disclosed herein in a high-throughput screening, and/or for stimulating neurons.

### DETAILED DESCRIPTION OF THE PREFERRED EMBODIMENTS

The present disclosure pertains to a mutant light-inducible ion channel, wherein the mutant light-inducible ion channel comprises an amino acid sequence which has at least 84% similarity/homology and/or 75% identity to the full length sequence of SEQ ID NO: 1 (ChR-2), and wherein the mutant light-inducible ion channel only differs from its parent light-inducible ion channel by a substitution at a position corresponding to F219 in SEQ ID NO: 1,

which substitution increases the off-kinetics of the mutant channel as compared to the parent channel, when compared by patch-clamp measurements in the whole cell configuration at a clamp potential of −60 mV, a bath solution of 140 mM NaCl, 2 mM CaCl₂, 2 MgCl₂, 10 mM HEPES, pH 7.4, and a pipette solution of 110 mM NaCl, 2 mM MgCl₂, 10 mM EGTA, 10 mM HEPES, pH 7.4, as defined in the claims.

Preferably, the off-kinetics of the mutant channel are measured in NG108-15 cells each heterologously expressing the mutant or parent channel. Successful protein expression can be proven, for example, by EGFP- or YFP-mediated fluorescence. Generally, photocurrents are measured in response to 500 ms light pulses with an intensity of 23 mW/mm² and a wavelength of 594 nm. The T_{off} value is determined by a fit of the current after cessation of illumination to a monoexponential function. In a preferred embodiment, the substitution is F219Y.

Preferably, said mutant light-inducible ion channel has at least 86%, preferably at least 88%, more preferably at least 90%, more preferably at least 92%, more preferably at least 94%, more preferably at least 96%, more preferably at least 98%, more preferably at least 99% similarity/homology to the full length of SEQ ID NO: 1 (ChR-2).

In addition, or alternatively, the mutant light-inducible ion channel has at least 80%, preferably at least 85%, more preferably at least 90%, more preferably at least 95%, more preferably at least 96%, more preferably at least 97%, more preferably at least 98%, more preferably at least 99% identity to the full length of SEQ ID NO: 1 (ChR-2).

Examples of light-inducible ion channels, which have at least 66% similarity/homology or at least 52% identity to the full length of SEQ ID NO: 1 are the chimera ReaChR (accession number KF448069, SEQ ID NO: 3), the Volvox Channelrhodopsin VChR1 (accession number EU622855, SEQ ID NO: 2), and any other ortholog or allelic variant thereof.

Also contemplated are swap mutants of a light-inducible ion channel, in which helix 6 has been replaced by the helix-6 motif of SEQ ID NO: 4, e.g. Chrimson (SEQ ID NO: 6), or Chrimson CS (SEQ ID NO: 7) in which helix 6 is replaced by helix 6 of ChR-2 including the F219Y substitution, respectively (cf. SEQ ID NO: 8 and 9).

Such a swap mutant has typically at least 56% homology/similarity to the full length of SEQ ID NO: 1 (ChR-2), preferably at least 58%, more preferably at least 60%, more preferably at least 62%, and even more preferably at least 64% homology/similarity to the full length of SEQ ID NO: 1 (ChR-2).

In addition, or alternatively, such a swap mutant has typically at least 42%, preferably at least 44%, more preferably at least 46%, more preferably at least 48%, more preferably at least 50%, more preferably at least 52%, more preferably at least 54%, more preferably at least 56% identity to the full length of SEQ ID NO: 1 (ChR-2).

Accordingly, the present disclosure also describes a mutant light-inducible ion channel, wherein the mutant light-inducible ion channel comprises an amino acid sequence which has at least 66% similarity/homology to the full length sequence of SEQ ID NO: 2 (VChR1), and wherein the mutant light-inducible ion channel only differs from its parent light-inducible ion channel by a substitution at a position corresponding to F214 in SEQ ID NO: 2,

which substitution increases the off-kinetics of the mutant channel as compared to the parent channel, when compared by patch-clamp measurements in the whole cell configuration at a clamp potential of −60 mV, a bath solution of 140 mM NaCl, 2 mM CaCl₂, 2 MgCl₂, 10 mM HEPES, pH 7.4, and a pipette solution of 110 mM NaCl, 2 mM MgCl₂, 10 mM EGTA, 10 mM HEPES, pH 7.4, and as further described above. Preferably, the substitution is F214Y.

Preferably, said mutant light-inducible ion channel has at least 70%, preferably at least 72%, more preferably at least 74%, more preferably at least 76%, more preferably at least 78%, more preferably at least 80%, more preferably at least 82%, more preferably at least 84%, more preferably at least 86%, more preferably at least 88%, more preferably at least 90%, more preferably at least 92%, more preferably at least 94%, more preferably at least 96%, more preferably at least 98%, more preferably at least 99% similarity/homology to the full length of SEQ ID NO: 2 (VChR1).

In addition, or alternatively, the mutant light-inducible ion channel has at least 52%, preferably at least 54%, more preferably at least 56%, more preferably at least 58%, more preferably at least 60%, more preferably at least 65%, more preferably at least 70%, more preferably at least 75%, more preferably at least 80%, more preferably at least 85%, more preferably at least 90%, more preferably at least 95%, more preferably at least 96%, more preferably at least 97%, more preferably at least 98%, more preferably at least 99% identity to the full length of SEQ ID NO: 2 (VChR1).

Further contemplated are swap mutants of a light-inducible ion channel, in which helix 6 has been replaced by the helix-6 motif of SEQ ID NO: 4, e.g. Chrimson (SEQ ID NO: 6), or Chrimson CS (SEQ ID NO: 7) in which helix 6 is replaced by helix 6 of VChR1 including the F214Y substitution, respectively.

Such a swap mutant has typically at least 61% homology/similarity to the full length of SEQ ID NO: 2 (VChR1), preferably at least 62%, more preferably at least 64%, and even more preferably at least 66% homology/similarity to the full length of SEQ ID NO: 2 (VChR1).

In addition, or alternatively, such a swap mutant has typically at least 45%, preferably at least 46%, more preferably at least 48%, more preferably at least 50%, identity to the full length of SEQ ID NO: 2 (VChR1).

Also disclosed is a mutant light-inducible ion channel, wherein the mutant light-inducible ion channel comprises an amino acid sequence which has at least 66% similarity/homology to the full length sequence of SEQ ID NO: 3 (ReaChR), and wherein the mutant light-inducible ion channel only differs from its parent light-inducible ion channel by a substitution at a position corresponding to F259 in SEQ ID NO: 3,

which substitution increases the off-kinetics of the mutant channel as compared to the parent channel, when compared by patch-clamp measurements in the whole cell configuration at a clamp potential of −60 mV, a bath solution of 140 mM NaCl, 2 mM CaCl₂, 2 MgCl₂, 10 mM HEPES, pH 7.4, and a pipette solution of 110 mM NaCl, 2 mM MgCl₂, 10 mM EGTA, 10 mM HEPES, pH 7.4, and as further described above. Preferably, the substitution is F259Y.

Preferably, said mutant light-inducible ion channel has at least 70%, preferably at least 72%, more preferably at least 74%, more preferably at least 76%, more preferably at least 78%, more preferably at least 80%, more preferably at least 82%, more preferably at least 84%, more preferably at least 86%, more preferably at least 88%, more preferably at least 90%, more preferably at least 92%, more preferably at least 94%, more preferably at least 96%, more preferably at least 98%, more preferably at least 99% similarity/homology to the full length of SEQ ID NO: 3 (ReaChR).

In addition, or alternatively, the mutant light-inducible ion channel has at least 52%, preferably at least 54%, more preferably at least 56%, more preferably at least 58%, more preferably at least 60%, more preferably at least 65%, more preferably at least 70%, more preferably at least 75%, more preferably at least 80%, more preferably at least 85%, more preferably at least 90%, more preferably at least 95%, more preferably at least 96%, more preferably at least 97%, more preferably at least 98%, more preferably at least 99% identity to the full length of SEQ ID NO: 3 (ReaChR).

Further contemplated are swap mutants of a light-inducible ion channel, in which helix 6 has been replaced by the helix-6 motif of SEQ ID NO: 4, e.g. Chrimson (SEQ ID NO: 6), or Chrimson CS (SEQ ID NO: 7) in which helix 6 is replaced by helix 6 of ReaChR including the F259Y substitution, respectively.

Such a swap mutant has typically at least 59% homology/similarity to the full length of SEQ ID NO: 3 (ReaChR), preferably at least 60%, more preferably at least 62%, and even more preferably at least 64% homology/similarity to the full length of SEQ ID NO: 3 (ReaChR).

In addition, or alternatively, such a swap mutant has typically at least 46%, preferably at least 48%, more preferably at least 50%, more preferably at least 52%, more preferably at least 54%, more preferably at least 56%, more preferably at least 58%, more preferably at least 60% identity to the full length of SEQ ID NO: 3 (ReaChR).

Generally, an amino acid sequence has “at least x % identity” with another amino acid sequence, e.g. SEQ ID NO: 1 above, when the sequence identity between those to aligned sequences is at least x % over the full length of said other amino acid sequence, e.g. SEQ ID NO: 1. Similarly, an amino acid sequence has “at least x % similarity/homology” with another amino acid sequence, e.g. SEQ ID NO: 1 above, when the sequence homology/sequence similarity between those to aligned sequences is at least x % over the full length of said other amino acid sequence, e.g. SEQ ID NO: 1.

Such alignments can be performed using for example publicly available computer homology programs such as the “EMBOSS” program provided at the EMBL homepage at http://www.ebi.ac.uk/Tools/psa/emboss_needle/, using the default settings provided therein. Further methods of calculating sequence identity or sequence similarity/homology percentages of sets of amino acid acid sequences are known in the art.

The light inducible ion channel of the present disclosure is a membrane protein with at least 5 transmembrane helices, which is capable of binding a light-sensitive polyene. Transmembrane proteins with 6 or 7 transmembrane helices are preferable. Transmembrane proteins with more than 7 helices, for example 8, 9 or 10 transmembrane helices, are however also encompassed. Furthermore, the invention covers transmembrane proteins which in addition to the transmembrane part include C- and/or N-terminal sequences, where the C-terminal sequences can extend into the inside of the lumen enclosed by the membrane, for example the cytoplasm of a cell or the inside of a liposome, or can also be arranged on the membrane outer surface. The same applies for the optionally present N-terminal sequences, which can likewise be arranged both within the lumen and also on the outer surface of the membrane. The length of the C- and/or N-terminal sequences is in principle subject to no restriction; however, light-inducible ion channels with C-terminal sequences not embedded in the membrane, with 1 to 1000 amino acids, preferably 1 to 500, especially preferably 5 to 50 amino acids, are preferred. Independently of the length of the C-terminal sequences, the N-terminal located sequences not embedded in the membrane preferably comprise 1 to 500 amino acids, especially preferably 5 to 50 amino acids. The concept of the transmembrane helix is well known to the skilled person. These are generally α-helical protein structures, which as a rule comprise 20 to 25 amino acids. However, depending on the nature of the membrane, which can be a natural membrane, for example a cell or plasma membrane, or also a synthetic membrane, the transmembrane segments can also be shorter or longer. For example, transmembrane segments in artificial membranes can comprise up to 30 amino acids, but on the other hand also only a few amino acids, for example 12 to 16.

In addition, the light-inducible ion channel comprises further (semi-)conservative substitutions as compared to SEQ ID NO: 1. Conservative substitutions are those that take place within a family of amino acids that are related in their side chains and chemical properties. Examples of such families are amino acids with basic side chains, with acidic side chains, with non-polar aliphatic side chains, with non-polar aromatic side chains, with uncharged polar side chains, with small side chains, with large side chains etc. Typical semi-conservative and conservative substitutions are:

| | Conservative | |
|---|---|---|
| Amino acid | substitution | Semi-conservative substitution |
| A | G; S; T | N; V; C |
| C | A; V; L | M; I; F; G |
| D | E; N; Q | A; S; T; K; R; H |
| E | D; Q; N | A; S; T; K; R; H |
| F | W; Y; L; M; H | I; V; A |
| G | A | S; N; T; D; E; N; Q |
| H | Y; F; K; R | L; M; A |
| I | V; L; M; A | F; Y; W; G |
| K | R; H | D; E; N; Q; S; T; A |
| L | M; I; V; A | F; Y; W; H; C |
| M | L; I; V; A | F; Y; W; C; |
| N | Q | D; E; S; T; A; G; K; R |
| P | V;I | L; A; M; W; Y; S; T; C; F |
| Q | N | D; E; A; S; T; L; M; K; R |
| R | K; H | N; Q; S; T; D; E; A |
| S | A; T; G; N | D; E; R; K |
| T | A; S; G; N; V | D; E; R; K; I |
| V | A; L; I | M; T; C; N |
| W | F; Y: H | L; M; I; V; C |
| Y | F; W; H | L; M; I; V; C |

Furthermore, the skilled person will appreciate that glycines at sterically demanding positions should not be substituted and that proline should not be introduced into parts of the protein which have an alpha-helical or a beta-sheet structure.

Preferably, the mutant channel comprises the motif of SEQ ID NO: 4:

Cys-Arg-Xaa₃-Xaa₄-Val-Xaa₆-Xaa₇-Met-Ala-Trp-Xaa₁₁-
Tyr-Phe-Val-Xaa₁₅-Trp-Gly-Met-Phe-Pro-Xaa₂₁-Leu-Phe-
Xaa₂₄-Leu,

wherein Xaa₃ is Gin or Glu, preferably wherein Xaa₃ is Gin;
wherein Xaa₄ is Val or Leu, preferably wherein Xaa₄ is Val;
wherein Xaa₆ is Thr or Arg, preferably wherein Xaa₆ is Thr;
wherein Xaa₇ is Gly, Val or Ala, preferably wherein Xaa₇ is Gly;
wherein Xaa₁₁ is Leu or Thr, preferably wherein Xaa₁₁ is Leu;
wherein Xaa₁₅ is Ser or Ala, preferably wherein Xaa₁₅ is Ser;
wherein Xaa₂₁ is Ile or Val, preferably wherein Xaa₂₁ is Ile; and
wherein Xaa₂₄ is Ile or Leu, preferably wherein Xaa₂₄ is Ile.

It is further preferred that the light-inducible ion channel comprises the consensus motif L(I,A,C)DxxxKxxW(F,Y) (SEQ ID NO: 5). Amino acids given in brackets can in each case replace the preceding amino acid. This consensus sequence is the motif surrounding the retinal-binding amino acid lysine.

In general, the retinal or retinal derivative necessary for the functioning of the light-inducible ion channel is produced by the cell to be transfected with said ion channel. Depending on its conformation, the retinal may be all-trans retinal, 11-cis-retinal, 13-cis-retinal, or 9-cis-retinal. However, it is also contemplated that the mutant light-inducible ion channel of the invention may be incorporated into vesicles, liposomes or other artificial cell membranes. Accordingly, also disclosed is a channelrhodopsin, comprising the light-inducible ion channel of the present disclosure, and a retinal or retinal derivative. Preferably, the retinal derivative is selected from the group consisting of 3,4-dehydroretinal, 13-ethylretinal, 9-dm-retinal, 3-hydroxyretinal, 4-hydroxyretinal, naphthylretinal; 3,7,11-trimethyl-dodeca-2,4,6,8,10-pentaenal; 3,7-dimethyl-deca-2,4,6,8-tetraenal; 3,7-dimethyl-octa-2,4,6-trienal; and 6-7 rotation-blocked retinals, 8-9 rotation-blocked retinals, and 10-11 rotation-blocked retinals.

As implicated above, the mutant light-inducible ion channel may additionally comprise further substitutions. In one preferred embodiment, the mutant light-inducible ion channel may additionally comprise Cys, Ser, Glu, Asp, or Thr, preferably Cys, at a position corresponding to position 132 of SEQ ID NO: 1. Said residues have been shown to increase the calcium permeability of the light-inducible ion channel.

The foregoing substitution is of particular interest, since the substitution of the present disclosure at the position corresponding to position 219 in SEQ ID NO: 1, position 214 in SEQ ID NO: 2, or position 259 in SEQ ID NO: 3 is shown herein to further increase the calcium permeability of the mutant light-inducible ion channel as compared to the parent.

For example, this can be measured by assessing the permeability of calcium ions relative to the permeability of sodium ions (P_{Ca}/P_{Na}), by measuring photocurrent-voltage relationships and determining the reversal potential. The relative calcium permeabilites are preferably determined in HEK293 cells, or NG108-15 cells. The intracellular solution contains 110 mM NaCl, 10 mM EGTA, 2 mM MgCl₂ and 10 mM Tris (pH=7.4) and the extracellular solution contains 140 mM NaCl, 2 mM MgCl₂ and 10 mM Tris (pH=9). For the determination of the P_{Ca}/P_{Na} values, external 140 mM NaCl is exchanged with 90 mM CaCl₂. Permeability ratios are calculated according to the Goldman-Hodgkin-Katz equation.¹⁵

Alternatively, the calcium conductivity of the mutant light-inducible ion channel of the invention is increased when compared to the parent channel, as determined by Fura-2-imaging on HEK293 cells. In order to determine the calcium conductivity, diflunisalFura-2 AM (5 mM; Invitrogen) is loaded at room temperature for 30 min to 1 hour. After loading, the cells are recovered in a 140 mM NaCl solution without Ca²⁺ (140 mM NaCl, 7 mM EGTA, 2 mM MgCl₂ and 10 mM HEPES). Yellow fluorescent protein is excited by a 500 ms exposure to light using a 460/40 nm filter (Visitron Systems, Puchheim, Germany) to estimate each cell's expression level from its YFP-fluorescence. The solution is then replaced by an extracellular Ca²⁺-solution that consists of 90 mM CaCl₂, 7 mM EGTA, 2 mM MgCl₂ and 10 mM HEPES. After 15 min in the dark the light-gated channels are stimulated for 10 s with blue light (460/40 nm). Fura-2 is excited with 340 nm (340/20) and 380 nm (380/20) and the emitted light (540/80 nm) detected with a CCD camera (all filters from Visitron Systems, Puchheim, Germany).

In addition, the mutant light-inducible ion channel may additionally comprise at least one of the following amino acid residues: aspartic acid at a position corresponding to position 253 of SEQ ID NO: 1; lysine at a position corresponding to position 257 of SEQ ID NO: 1; tryptophan at a position corresponding to position 260 of SEQ ID NO: 1; glutamic acid at a position corresponding to position 123 of SEQ ID NO: 1; histidine or arginine, preferably arginine, at a position corresponding to position 134 of SEQ ID NO: 1; threonine, serine, or alanine at a position corresponding to position 128 of SEQ ID NO: 1; and/or alanine at a position corresponding to position 156 of SEQ ID NO: 1. Accordingly, the mutant light-inducible ion channel may additionally comprise one of the following combinations of amino acid residues at the indicated positions, which positions correspond to SEQ ID NO: 1:

### Cys 132+Asp 253; Cys 132+Lys 257; Cys 132+Trp 260; Cys 132+Glu 123; Cys 132+His 134; Cys 132+Arg 134; Cys 132+Thr 128; Cys 132+Ser 128; Cys 132+Ala 128; Cys 132+Ala 156;

Cys 132+Asp 253+Lys 257; Cys 132+Asp 253+Trp 260; Cys 132+Asp 253+Glu 123; Cys 132+Asp 253+His 134; Cys 132+Asp 253+Arg 134; Cys 132+Asp 253+Thr 128; Cys 132+Asp 253+Ser 128; Cys 132+Asp 253+Ala 128; Cys 132+Asp 253+Ala 156;
Cys 132+Lys 257+Trp 260; Cys 132+Lys 257+Glu 123; Cys 132+Lys 257+His 134; Cys 132+Lys 257+Arg 134; Cys 132+Lys 257+Thr 128; Cys 132+Lys 257+Ser 128; Cys 132+Lys 257+Ala 128; Cys 132+Lys 257+Ala 156; Cys 132+Trp 260+Glu 123; Cys 132+Trp 260+His 134; Cys 132+Trp 260+Arg 134; Cys 132+Trp 260+Thr 128; Cys 132+Trp 260+Ser 128; Cys 132+Trp 260+Ala 128; Cys 132+Trp 260+Ala 156;

### Cys 132+Glu 123+His 134; Cys 132+Glu 123+His 134; Cys 132+Glu 123+Arg 134; Cys 132+Glu 123+Thr 128; Cys 132+Glu 123+Ser 128; Cys 132+Glu 123+Ala 128; Cys 132+Glu 123+Ala 156;

### Cys 132+His 134+Thr 128; Cys 132+His 134+Ser 128; Cys 132+His 134+Ala 128; Cys 132+His 134+Ala 156;

### Cys 132+Arg 134+Thr 128; Cys 132+Arg 134+Ser 128; Cys 132+Arg 134+Ala 128; Cys 132+Arg 134+Ala 156;

### Cys 132+Thr 128+Ala 156; Cys 132+Ser 128+Ala 156; Cys 132+Ala 128+Ala 156;

### Cys 132+Asp 253+Lys 257+Trp 260; Cys 132+Asp 253+Lys 257+Glu 123;

### Cys 132+Asp 253+Lys 257+His 134; Cys 132+Asp 253+Lys 257+Arg 134;

### Cys 132+Asp 253+Lys 257+Thr 128; Cys 132+Asp 253+Lys 257+Ser 128;

### Cys 132+Asp 253+Lys 257+Ala 128; Cys 132+Asp 253+Lys 257+Ala 156;

### Cys 132+Lys 157+Trp 260+Glu 123; Cys 132+Lys 157+Trp 260+His 134;

### Cys 132+Lys 157+Trp 260+Arg 134; Cys 132+Lys 157+Trp 260+Thr 128;

### Cys 132+Lys 157+Trp 260+Ser 128; Cys 132+Lys 157+Trp 260+Ala 128;

### Cys 132+Lys 157+Trp 260+Ala 156;

### Cys 132+Trp 260+Glu 123+His 134; Cys 132+Trp 260+Glu 123+Arg 134;

### Cys 132+Trp 260+Glu 123+Thr 128; Cys 132+Trp 260+Glu 123+Ser 128;

### Cys 132+Trp 260+Glu 123+Ala 128; Cys 132+Trp 260+Glu 123+Ala 156;

### Cys 132+Glu 123+His 134+Thr 128; Cys 132+Glu 123+His 134+Ser 128;

### Cys 132+Glu 123+His 134+Ala 128; Cys 132+Glu 123+His 134+Ala 156;

### Cys 132+Glu 123+Arg 134+Thr 128; Cys 132+Glu 123+Arg 134+Ser 128;

### Cys 132+Glu 123+Arg 134+Ala 128; Cys 132+Glu 123+Arg 134+Ala 156;

### Cys 132+His 134+Thr 128+Ala 156; Cys 132+His 134+Ser 128+Ala 156; Cys 132+His 134+Ala 128+Ala 156;

### Cys 132+Arg 134+Thr 128+Ala 156; Cys 132+Arg 134+Ser 128+Ala 156; Cys 132+Arg 134+Ala 128+Ala 156;

Cys 132+Asp 253+Lys 257+Trp 260+Glu 123; Cys 132+Asp 253+Lys 257+Trp 260+His 134; Cys 132+Asp 253+Lys 257+Trp 260+Arg 134; Cys 132+Asp 253+Lys 257+Trp 260+Thr 128; Cys 132+Asp 253+Lys 257+Trp 260+Ser 128; Cys 132+Asp 253+Lys 257+Trp 260+Ala 128; Cys 132+Asp 253+Lys 257+Trp 260+Ala 156;
Cys 132+Lys 257+Trp 260+Glu 123+His 134; Cys 132+Lys 257+Trp 260+Glu 123+Arg 134; Cys 132+Lys 257+Trp 260+Glu 123+Thr 128; Cys 132+Lys 257+Trp 260+Glu 123+Ser 128; Cys 132+Lys 257+Trp 260+Glu 123+Ala 128; Cys 132+Lys 257+Trp 260+Glu 123+Ala 156;

### Cys 132+Trp 260+Glu 123+His 134+Thr 128; Cys 132+Trp 260+Glu 123+His 134+Ser 128; Cys 132+Trp 260+Glu 123+His 134+Ala 128; Cys 132+Trp 260+Glu 123+His 134+Ala 156;

### Cys 132+Trp 260+Glu 123+Arg 134+Thr 128; Cys 132+Trp 260+Glu 123+Arg 134+Ser 128; Cys 132+Trp 260+Glu 123+Arg 134+Ala 128; Cys 132+Trp 260+Glu 123+Arg 134+Ala 156;

### Cys 132+Glu 123+Arg 134+Thr 128+Ala 156; Cys 132+Glu 123+Arg 134+Ser 128+Ala 156; Cys 132+Glu 123+Arg 134+Ala 128+Ala 156;

### Cys 132+Glu 123+His 134+Thr 128+Ala 156; Cys 132+Glu 123+His 134+Ser 128+Ala 156; Cys 132+Glu 123+His 134+Ala 128+Ala 156;

Cys 132+Asp 253+Lys 257+Trp 260+Glu 123+His 134; Cys 132+Asp 253+Lys 257+Trp 260+Glu 123+Arg 134; Cys 132+Asp 253+Lys 257+Trp 260+Glu 123+Thr 128; Cys 132+Asp 253+Lys 257+Trp 260+Glu 123+Ser 128; Cys 132+Asp 253+Lys 257+Trp 260+Glu 123+Ala 128; Cys 132+Asp 253+Lys 257+Trp 260+Glu 123+Ala 156;
Cys 132+Lys 257+Trp 260+Glu 123+His 134+Thr 128; Cys 132+Lys 257+Trp 260+Glu 123+His 134+Ser 128; Cys 132+Lys 257+Trp 260+Glu 123+His 134+Ala 128; Cys 132+Lys 257+Trp 260+Glu 123+His 134+Ala 156;
Cys 132+Lys 257+Trp 260+Glu 123+Arg 134+Thr 128; Cys 132+Lys 257+Trp 260+Glu 123+Arg 134+Ser 128; Cys 132+Lys 257+Trp 260+Glu 123+Arg 134+Ala 128; Cys 132+Lys 257+Trp 260+Glu 123+Arg 134+Ala 156;

### Cys 132+Trp 260+Glu 123+Arg 134+Thr 128+Ala 156; Cys 132+Trp 260+Glu 123+Arg 134+Ser 128+Ala 156; Cys 132+Trp 260+Glu 123+Arg 134+Ala 128+Ala 156;

### Cys 132+Trp 260+Glu 123+His 134+Thr 128+Ala 156; Cys 132+Trp 260+Glu 123+His 134+Ser 128+Ala 156; Cys 132+Trp 260+Glu 123+His 134+Ala 128+Ala 156;

Cys 132+Asp 253+Lys 257+Trp 260+Glu 123+His 134+Thr 128; Cys 132+Asp 253+Lys 257+Trp 260+Glu 123+His 134+Ser 128; Cys 132+Asp 253+Lys 257+Trp 260+Glu 123+His 134+Ala 128; Cys 132+Asp 253+Lys 257+Trp 260+Glu 123+His 134+Ala 156;
Cys 132+Asp 253+Lys 257+Trp 260+Glu 123+Arg 134+Thr 128; Cys 132+Asp 253+Lys 257+Trp 260+Glu 123+Arg 134+Ser 128; Cys 132+Asp 253+Lys 257+Trp 260+Glu 123+Arg 134+Ala 128; Cys 132+Asp 253+Lys 257+Trp 260+Glu 123+Arg 134+Ala 156;
Cys 132+Lys 257+Trp 260+Glu 123+His 134+Thr 128+Ala 156; Cys 132+Lys 257+Trp 260+Glu 123+His 134+Ser 128+Ala 156; Cys 132+Lys 257+Trp 260+Glu 123+His 134+Ala 128+Ala 156;
Cys 132+Lys 257+Trp 260+Glu 123+Arg 134+Thr 128+Ala 156; Cys 132+Lys 257+Trp 260+Glu 123+Arg 134+Ser 128+Ala 156; Cys 132+Lys 257+Trp 260+Glu 123+Arg 134+Ala 128+Ala 156;

### Cys 132+Asp 253+Lys 257+Trp 260+Glu 123+His 134+Thr 128+Ala 156;

### Cys 132+Asp 253+Lys 257+Trp 260+Glu 123+His 134+Ser 128+Ala 156;

### Cys 132+Asp 253+Lys 257+Trp 260+Glu 123+His 134+Ala 128+Ala 156;

### Cys 132+Asp 253+Lys 257+Trp 260+Glu 123+Arg 134+Thr 128+Ala 156;

### Cys 132+Asp 253+Lys 257+Trp 260+Glu 123+Arg 134+Ser 128+Ala 156;

### Cys 132+Asp 253+Lys 257+Trp 260+Glu 123+Arg 134+Ala 128+Ala 156.

However, in the above list, Cys 132 may also be substituted by either Ser 132, Glu 132, Asp 132, or Thr 132.

In a preferred embodiment, the mutant light-inducible ion channel comprises, preferably consists of the amino acid sequence of SEQ ID NO: 1 (ChR-2), except for said substitution at position F219, and optionally the amino acid at position 132 of SEQ ID NO: 1.

Likewise, in another preferred embodiment, the mutant light-inducible ion channel comprises, preferably consists of the amino acid sequence of SEQ ID NO: 2 (VChR1), except for said substitution at position F214, and optionally the amino acid at the position in SEQ ID NO: 2 corresponding to L132 in SEQ ID NO: 1.

In still another preferred embodiment, the mutant light-inducible ion channel comprises, preferably consists of the amino acid sequence of SEQ ID NO: 3 (ReaChR), except for said substitution at position F259, and optionally the amino acid at the position in SEQ ID NO: 3 corresponding to L132 in SEQ ID NO: 1.

The present disclosure also describes a nucleic acid construct, comprising a nucleotide sequence coding for the mutant light-inducible ion channel as disclosed herein above.

To ensure optimal expression, the coding DNA can also be suitably modified, for example by adding suitable regulatory sequences and/or targeting sequences and/or by matching of the coding DNA sequence to the preferred codon usage of the chosen host. The targeting sequence may encode a C-terminal extension targeting the light-inducible ion channel to a particular site or compartment within the cell, such as to the synapse or to a post-synaptic site, to the axon-hillock, or the endoplasmic reticulum. The nucleic acid may be combined with further elements, e.g., a promoter and a transcription start and stop signal and a translation start and stop signal and a polyadenylation signal in order to provide for expression of the sequence of the mutant light-inducible ion channel of the present disclosure. The promoter can be inducible or constitutive, general or cell specific promoter. An example of a cell-specific promoter is the mGlu6-promotor specific for bipolar cells. Selection of promoters, vectors and other elements is a matter of routine design within the level of ordinary skill in the art. Many such elements are described in the literature and are available through commercial suppliers.

Also disclosed is an expression vector, comprising a nucleotide sequence coding for the mutant light-inducible ion channel or the nucleic acid construct as disclosed herein. In a preferred embodiment, the vector is suitable for gene therapy, in particular wherein the vector is suitable for virus-mediated gene transfer. The term “suitable for virus-mediated gene transfer” means herein that said vector can be packed in a virus and thus be delivered to the site or the cells of interest. Examples of viruses suitable for gene therapy are retroviruses, adenoviruses, adeno-associated viruses, lentiviruses, pox viruses, alphaviruses, rabies virus, semliki forest virus and herpes viruses. These viruses differ in how well they transfer genes to the cells they recognize and are able to infect, and whether they alter the cell's DNA permanently or temporarily. However, gene therapy also encompasses non-viral methods, such as application of naked DNA, lipoplexes and polyplexes, and dendrimers.

As described above, the resulting nucleic acid sequence may be introduced into cells e.g. using a virus as a carrier or by transfection including e.g. by chemical transfectants (such as Lipofectamine, Fugene, etc.), electroporation, calcium phosphate co-precipitation and direct diffusion of DNA. A method for transfecting a cell is detailed in the examples and may be adapted to the respective recipient cell. Transfection with DNA yields stable cells or cell lines, if the transfected DNA is integrated into the genome, or unstable (transient) cells or cell lines, wherein the transfected DNA exists in an extrachromosomal form. Furthermore, stable cell lines can be obtained by using episomal replicating plasmids, which means that the inheritance of the extrachromosomal plasmid is controlled by control elements that are integrated into the cell genome. In general, the selection of a suitable vector or plasmid depends on the intended host cell.

Therefore, the present disclosure also pertains to a cell comprising the nucleic acid construct or the expression vector as disclosed herein.

The incorporation of the mutant light-inducible ion channel into the membrane of cells which do not express the corresponding channels in nature can for example be simply effected in that, using known procedures of recombinant DNA technology, the DNA coding for this ion channel is firstly incorporated into a suitable expression vector, e.g. a plasmid, a cosmid or a virus, the target cells are then transformed with this, and the protein is expressed in this host. Next, the cells are treated in a suitable manner, e.g. with retinal, in order to enable the linkage of a Schiffs base between protein and retinal.

The expression of the light-inducible ion channel of the present disclosure can be advantageously effected in certain mammalian cell systems. Thus, in a preferred embodiment, the cell is a mammalian cell. The expression is effected either with episomal vectors as transient expression, preferably in neuroblastoma cells (e.g., NG108-15-Cells), melanoma cells (e.g., the BLM cell line), COS cells (generated by infection of “African green monkey kidney CV1” cells) or HEK cells (“human embryonic kidney cells”, e.g. HEK293 cells), or BHK-cells (“baby hamster kidney cells”), or in the form of stable expression (by integration into the genome) in CHO cells (“Chinese hamster ovary cells”), myeloma cells or MDCK cells (“Madine-Darby canine kidney cells”) or in Sf9 insect cells infected with baculoviruses. Accordingly, in a more preferred embodiment the mammalian cell is a COS cell; a BHK cell; a HEK293 cell; a CHO cell; a myeloma cell; or a MDCK cell.

In a preferred embodiment, the mammalian cell is an electrically excitable cell. It is further preferred that the cell is a hippocampal cell, a photoreceptor cell; a retinal rod cell; a retinal cone cell; a retinal ganglion cell; a bipolar neuron; a ganglion cell; a pseudounipolar neuron; a multipolar neuron; a pyramidal neuron, a Purkinje cell; or a granule cell.

A neuron is an electrically excitable cell that processes and transmits information by electrical and chemical signalling, wherein chemical signaling occurs via synapses, specialized connections with other cells. A number of specialized types of neurons exist such as sensory neurons responding to touch, sound, light and numerous other stimuli affecting cells of the sensory organs, motor neurons receiving signals from the brain and spinal cord and causing muscle contractions and affecting glands, and interneurons connecting neurons to other neurons within the same region of the brain or spinal cord. Generally, a neuron possesses a soma, dendrites, and an axon. Dendrites are filaments that arise from the cell body, often extending for hundreds of microns and branching multiple times. An axon is a special cellular filament that arises from the cell body at a site called the axon hillock. The cell body of a neuron frequently gives rise to multiple dendrites, but never to more than one axon, although the axon may branch hundreds of times before it terminates. At the majority of synapses, signals are sent from the axon of one neuron to a dendrite of another. There are, however, many exceptions to these rules: neurons that lack dendrites, neurons that have no axon, synapses that connect an axon to another axon or a dendrite to another dendrite, etc. Most neurons can further be anatomically characterized as unipolar or pseudounipolar (dendrite and axon emerge from same process), bipolar (axon and single dendrite on opposite ends of the soma), multipolar (having more than two dendrites and may be further classified as (i) Golgi I neurons with long-projecting axonal processes, such as pyramidal cells, Purkinje cells, and anterior horn cells, and (ii) Golgi II: neurons whose axonal process projects locally, e.g., granule cells.

A photoreceptor cell, is a specialized neuron found in the retina that is capable of phototransduction. The two classic photoreceptors are rods and cones, each contributing information used by the visual system. A retinal ganglion cell is a type of neuron located near the inner surface of the retina of the eye. These cells have dendrites and long axons projecting to the protectum (midbrain), the suprachiasmatic nucleus in the hypothalamus, and the lateral geniculate (thalamus). A small percentage contribute little or nothing to vision, but are themselves photosensitive. Their axons form the retinohypothalamic tract and contribute to circadian rhythms and pupillary light reflex, the resizing of the pupil. They receive visual information from photoreceptors via two intermediate neuron types: bipolar cells and amacrine cells. Amacrine cells are interneurons in the retina, and responsible for 70% of input to retinal ganglion cells. Bipolar cells, which are responsible for the other 30% of input to retinal ganglia, are regulated by amacrine cells. As a part of the retina, the bipolar cell exists between photoreceptors (rod cells and cone cells) and ganglion cells. They act, directly or indirectly, to transmit signals from the photoreceptors to the ganglion cells.

The cell may be isolated (and genetically modified), maintained and cultured at an appropriate temperature and gas mixture (typically, 37° C., 5% CO02), optionally in a cell incubator as known to the skilled person and as exemplified for certain cell lines or cell types in the examples. Culture conditions may vary for each cell type, and variation of conditions for a particular cell type can result in different phenotypes. Aside from temperature and gas mixture, the most commonly varied factor in cell culture systems is the growth medium. Recipes for growth media can vary in pH, glucose concentration, growth factor and the presence of other nutrient components among others. Growth media are either commercially available, or can be prepared according to compositions, which are obtainable from the American Tissue Culture Collection (ATCC). Growth factors used for supplement media are often derived from animal blood such as calf serum. Additionally, antibiotics may be added to the growth media. Amongst the common manipulations carried out on culture cells are media changes and passaging cells. The present disclosure further pertains to a use of a mutant light-inducible ion channel, or a cell according to the present disclosure in a high-throughput screening. A high-throughput screening (HTS), is a method for scientific experimentation especially used in drug discovery and relevant to the fields of biology and chemistry. HTS allows a researcher to effectively conduct millions of biochemical, genetic or pharmacological tests in a short period of time, often through a combination of modern robotics, data processing and control software, liquid handling devices, and sensitive detectors. By this process, one may rapidly identify active agents which modulate a particular biomolecular pathway; particularly a substance modifying an ion channel, such as the light-inducible ion channel according to the invention, a Ca²⁺-inducible potassium channel, or a BK channel. For example, one might co-express the Ca²⁺-inducible potassium channel and the light-inducible ion channel in a host cell. Upon stimulation by light, the light-inducible channel will open and the intracellular Ca²⁺ concentration will increase, thereby activating the potassium channel. Thus, one will receive a change in the membrane potential, which may be monitored by potential-sensitive dyes such as RH 421 (N-(4-Sulfobutyl)-4-(4-(4-(dipentylamino)phenyl)butadienyl)pyridinium, inner salt). Such a HTS may thus comprise the following steps: (i) contacting a cell expressing a Ca²⁺-inducible (potassium) channel and the light-inducible ion channel according to the invention with a candidate agent directed against the Ca²⁺-inducible channel, (ii) applying a light stimulus in order to induce the light-inducible channel, (iii) determining the alteration of the membrane potential (mixed signal), and (iv) comparing the signal determined in step (iii) with the signal determined in a cell only expressing the light-inducible ion channel according to the invention subjected to step (ii) (single signal). A reduction in the change of the membrane potential would be indicative of a promising modulator of the Ca²⁺-inducible (potassium) channel. Such an approach is supposed to yield a signal-to-noise ratio of approximately 5:1, which is quite improved compared to direct measurements conducted on a cell only expressing the Ca²⁺-inducible channel. Due to the improved signal-to-noise ratio, said method, in particular by using the light-inducible ion channel, may be particularly suitable for HTS.

In essence, HTS uses an approach to collect a large amount of experimental data on the effect of a multitude of substances on a particular target in a relatively short time. A screen, in this context, is the larger experiment, with a single goal (usually testing a scientific hypothesis), to which all this data may subsequently be applied. For HTS cells according to the invention may be seed in a tissue plate, such as a multi well plate, e.g. a 96-well plate. Then the cell in the plate is contacted with the test substance for a time sufficient to interact with the targeted ion channel. The test substance may be different from well to well across the plate. After incubation time has passed, measurements are taken across all the plate's wells, either manually or by a machine and optionally compared to measurements of a cell which has not been contacted with the test substance. Manual measurements may be necessary when the researcher is using patch-clamp, looking for effects not yet implemented in automated routines. Otherwise, a specialized automated analysis machine can run a number of experiments on the wells (such as analysing light of a particular frequency or a high-throughput patch-clamp measurement). In this case, the machine outputs the result of each experiment e.g. as a grid of numeric values, with each number mapping to the value obtained from a single well. Depending upon the results of this first assay, the researcher can perform follow up assays within the same screen by using substances similar to those identified as active (i.e. modifying an intracellular cyclic nucleotide level) into new assay plates, and then re-running the experiment to collect further data, optimize the structure of the chemical agent to improve the effect of the agent on the cell. Automation is an important element in HTS's usefulness. A specialized robot is often responsible for much of the process over the lifetime of a single assay plate, from creation through final analysis. An HTS robot can usually prepare and analyze many plates simultaneously, further speeding the data-collection process. Examples for apparatuses suitable for HTS in accordance with the present invention comprise a Fluorometric Imaging Plate Reader (FLIPR™; Molecular Devices), FLEXstation™ (Molecular Devices), Voltage Ion Probe Reader (VIPR, Aurora Biosciences), Attofluor® Ratio Vision® (ATTO).

Thus, the presently disclosed mutant light-inducible ion channel is particularly useful as a research tool, such as in a non-therapeutic use for light-stimulation of electrically excitable cells, in particular neuron cells. Further guidance, e.g., with regard to Hippocampal neuron culture, and electrophysiological recordings from hippocampal neurons, as well as electrophysiological recordings on HEK293 cells, can be found in WO 2012/032103.

Finally, there are a number of diseases in which, e.g., the natural visual cells no longer function, but all nerve connections are capable of continuing to operate. Today, attempts are being made in various research centres to implant thin films with artificial ceramic photocells on the retina. These photocells are intended to depolarise the secondary, still intact cells of the retinal and thereby to trigger a nerve impulse (bionic eyes). The deliberate expression of mutant light-controlled ion channels according to the present disclosure in these ganglion cells, amacrine cells or bipolar cells would be a very much more elegant solution and enable greater three-dimensional visual resolution. Therefore, the present disclosure also contemplates the light-inducible ion channel according to the present disclosure for use in medicine. As shown in the examples below, the proof of principle was already demonstrated in the art, and can easily be adapted to the presently disclosed light-inducible ion channels. In view of these data, it is contemplated that the presently disclosed light-inducible ion channels can be used for restoring auditory activity in deaf subjects, or recovery of vision in blind subjects.

Further described are non-human animals which functionally express the light-inducible ion channel according to the present disclosure, e.g. in an electrically excitable cell such as a neuron, in particular in spiral ganglion neurons, as also described for the cell of the present disclosure. Likewise, also contemplated are non-human animals, which comprise a cell according to the present disclosure.

The non-human animal may be any animal other than a human. In a preferred embodiment, the non-human animal is a vertebrate, preferably a mammal, more preferably a rodent, such as a mouse or a rat, or a primate.

In particular, some model organisms are preferred, such as Caenorhabditis elegans, Arbacia punctulata, Ciona intestinalis, Drosophila, usually the species Drosophila melanogaster, Euprymna scolopes, Hydra, Loligo pealei, Pristionchus pacificus, Strongylocentrotus purpuratus, Symsagittifera roscoffensis, and Tribolium castaneum. Among vertebrates, these are several rodent species such as guinea pig (Cavia porcellus), hamster, mouse (Mus musculus), and rat (Rattus norvegicus), as well as other species such as chicken (Gallus gallus domesticus), cat (Felis cattus), dog (Canis lupus familiaris), Lamprey, Japanese ricefish (Oryzias latipes), Rhesus macaque, Sigmodon hispidus, zebra finch (Taeniopygia guttata), pufferfish (Takifugu rubripres), african clawed frog (Xenopus laevis), and zebrafish (Danio rerio). Also preferred are non-human primates, i.e. all species of animals under the order Primates that are not a member of the genus Homo, for example rhesus macaque, chimpanzee, baboon, marmoset, and green monkey. However, these examples are not intended to limit the scope of the invention.

However, it is noted that those animals are excluded, which are not likely to yield in substantial medical benefit to man or animal and which are therefore not subject to patentability under the respective patent law or jurisdiction. Moreover, the skilled person will take appropriate measures, as e.g. laid down in international guidelines of animal welfare, to ensure that the substantial medical benefit to man or animal will outweigh any animal suffering. In the following, the present invention is illustrated by figures and examples which are not intended to limit the scope of the present invention.

### DESCRIPTION OF THE FIGURES

FIG. 1: Alignment of the helix 6 region of ChR2, VChR1, and ReaChR. Also shown is the percentage identity and percentage similarity/homology of ChR2 (SEQ ID NO: 1), VChR1 (SEQ ID NO: 2), and ReaChR (SEQ ID NO: 3) over the full length of ChR2.

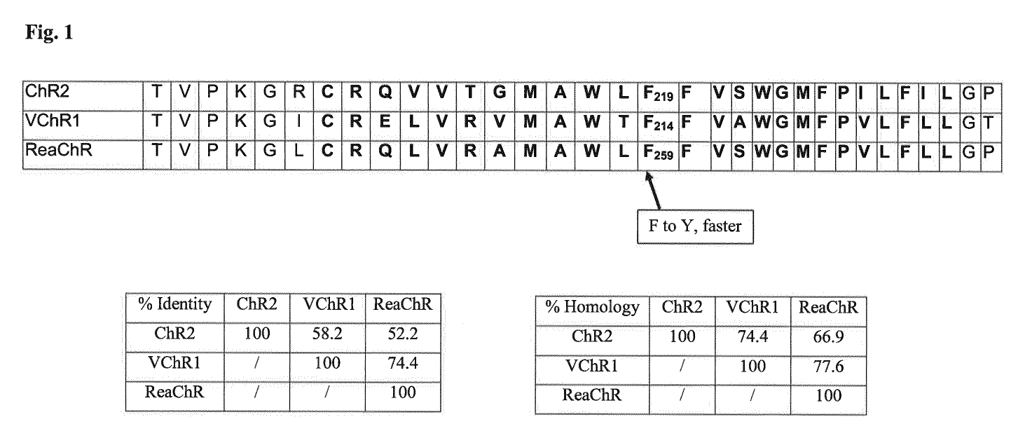

FIG. 2: Off-kinetics of Channelrhodopsin variants. Shown are typical photo currents of A) ChR2-YFP (right graph) and ChR2-YFP F219Y (left graph), B) VChR1-YFP (right graph) and VChR1-YFP F214Y (left graph), C) ReaChR-Citrine (right graph) and ReaChR-Citrine F259Y (left graph), immediately after cessation of illumination. The 0.5 s light pulses had a saturing light intensity of 23 mW/mm² and a wavelength of A) A=473 nm B) λ=532 nm C) λ=532 nm. NG108-15 cells which were heterologously expressing the corresponding Channelrhodopsin variant were investigated by patch-clamp measurements in the whole cell configuration at a clamped potential of −60 mV. The bath solution contained 140 mM NaCl, 2 mM CaCl₂, 2 mM MgCl₂, 10 mM HEPES, pH 7.4 and the pipette solution contained 110 mM NaCl, 2 mM MgCl₂, 10 mM EGTA, 10 mM HEPES, pH 7.4. The currents were normalized for comparison.

DESCRIPTION OF THE SEQUENCES

SEQ ID NO: 1 (Channelrhodopsin 2; ChR2; 315 aa; helix 6 highlighted
in bold)
MDYGGALSAVGRELLFVTNPVVVNGSVLVPEDQCYCAGWIESRGTNGAQTASN
VLQWLAAGFSILLLMFYAYQTWKSTCGWEEIYVCAIEMVKVILEFFFEFKNPSMLY
LATGHRVQWLRYAEWLLTCPVILIHLSNLTGLSNDYSRRIMGLLVSDIGTIVWGA
TSAMATGYVKVIFFCLGLCYGANTFFHAAKAYIEGYHTVPKGRCRQVVTGMAWL
FFVSWGMFPILFILGPEGFGVLSVYGSTVGHTIIDLMSKNCWGLLGHYLRVLIHEH
ILIHGDIRKTTKLNIGGTEIEVETLVEDEAEAGAVNKGTGK
SEQ ID NO: 2 (VChR1; accession number EU622855; 300 aa; helix 6
highlighted in bold)
MDYPVARSLIVRYPTDLGNGTVCMPRGQCYCEGWLRSRGTSIEKTIAITLQWW
FALSVACLGWYAYQAWRATCGWEEVYVALIEMMKSIIEAFHEFDSPATLWLSSG
NGVVWMRYGEWLLTCPVLLIHLSNLTGLKDDYSKRTMGLLVSDVGCMA/GATSA
MCTGWTKILFFLISLSYGMYTYFHAAKVYIEAFHTVPKGICRELVRVMAWTFFVA
WGMFPVLFLLGTEGFGHISPYGSAIGHSILDLIAKNMWGVLGNYLRVKIHEHILLY
GDIRKKQKITIAGQEMEVETLVAEEED
SEQ ID NO: 3 (ReaChR; accession number KF448069; 352 aa; helix 6
highlighted in bold)
MVSRRPWLLALALAVALAAGSAGASTGSDATVPVATQDGPDYVFHRAHERMLF
QTSYTLENNGSVICIPNNGQCFCLAWLKSNGTNAEKLAANILQWWFALSVACLG
WYAYQAWRATCGWEEVYVALIEMMKSIIEAFHEFDSPATLWLSSGNGWWMRY
GEWLLTCPVILIHLSNLTGLKDDYSKRTMGLLVSDVGCIWVGATSAMCTGWTKIL
FFLISLSYGMYTYFHAAKVYIEAFHTVPKGLCRQLVRAMAWLFFVSWGMFPVLF
LLGPEGFGHISPYGSAIGHSILDLIAKNMWGVLGNYLRVKIHEHILLYGDIRKKQKI
TIAGQEMEVETLVAEEEDKYESSLE
SEQ ID NO: 4 (Helix 6 Consensus Motif)
Cys-Arg-Xaa₃-Xaa₄-Val-Xaa₆-Xaa₇-Met-Ala-Trp-Xaa₁₁-Tyr-Phe-Val-Xaa₁₅-
Trp-Gly-Met-Phe-Pro-Xaa₂₁-Leu-Phe-Xaa₂₄-Leu,

wherein Xaa₃ is Gin or Glu, preferably wherein Xaa₃ is Gin;
wherein Xaa₄ is Val or Leu, preferably wherein Xaa₄ is Val;
wherein Xaa₆ is Thr or Arg, preferably wherein Xaa₆ is Thr;
wherein Xaa₇ is Gly, Val or Ala, preferably wherein Xaa₇ is Gly;
wherein Xaa₁₁ is Leu or Thr, preferably wherein Xaa₁₁ is Leu;
wherein Xaa₁₅ is Ser or Ala, preferably wherein Xaa₁₅ is Ser;
wherein Xaa₂₁ is Ile or Val, preferably wherein Xaa₂₁ is Ile; and
wherein Xaa₂₄ is Ile or Leu, preferably wherein Xaa₂₄ is Ile.

(Retinal binding site consensus motif)
SEQ ID NO: 5
Xaa₁-Asp-Xaa₃-Xaa₄-Xaa₅-Lys-Xaa₇-Xaa₈-Xaa₉

wherein Xaa₁ is Leu, Ile, Ala, or Cys;
wherein Xaa₃, Xaa₄, Xaa₅, Xaa₇, and Xaa₈ is independently any amino acid;
wherein Xaa₉ is Thr, Phe, or Tyr.

(Chrimson; accession number KF992060; helix 6 highlighted in
bold)
SEQ ID NO: 6
MAELISSATRSLFAAGGINPWPNPYHHEDMGCGGMTPTGECFSTEWWCDPSY
GLSDAGYGYCFVEATGGYLVVGVEKKQAWLHSRGTPGEKIGAQVCQWIAFSIAI
ALLTFYGFSAWKATCGWEEVYVCCVEVLFVTLEIFKEFSSPATVYLSTGNHAYCL
RYFEWLLSCPVILIKLSNLSGLKNDYSKRTMGLIVSCVGMIVFGMAAGLATDWLK
WLLYIVSCIYGGYMYFQAAKCYVEANHSVPKGHCRMVVKLMAYAYFASWGSYP
ILWAVGPEGLLKLSPYANSIGHSICDIIAKEFWTFLAHHLRIKIHEHILIFIGDIRKTTK
EIGGEEVEVEEFVEEEDEDTV
(CsChrimson; accession number KJ995863; helix 6 highlighted
in bold)
SEQ ID NO: 7
MSRLVAASWLLALLLCGITSTTTASSAPAASSTDGTAAAAVSHYAMNGFDELAKG
AVVPEDHFVCGPADKCYCSAWLHSRGTPGEKIGAQVCQWIAFSIAIALLTFYGFS
AWKATCGWEEVYVCCVEVLFVTLEIFKEFSSPATVYLSTGNHAYCLRYFEWLLS
CPVILIKLSNLSGLKNDYSKRTMGLIVSCVGMIVFGMAAGLATDWLKWLLYIVSCIY
GGYMYFQAAKCYVEANHSVPKGHCRMVVKLMAYAYFASWGSYPILWAVGPEG
LLKLSPYANSIGHSICDIIAKEFWTFLAHHLRIKIHEHILIHGDIRKTTKMEIGGEEVE
VEEFVEEEDEDTV
(Chrimson Helix 6 swap mutant including F219Y)
SEQ ID NO: 8
MAELISSATRSLFAAGGINPWPNPYHHEDMGCGGMTPTGECFSTEWWCDPSY
GLSDAGYGYCFVEATGGYLVVGVEKKQAWLHSRGTPGEKIGAQVCQWIAFSIAI
ALLTFYGFSAWKATCGWEEVYVCCVEVLFVTLEIFKEFSSPATVYLSTGNHAYCL
RYFEWLLSCPVILIKLSNLSGLKNDYSKRTMGLIVSCVGMIVFGMAAGLATDWLK
WLLYIVSCIYGGYMYFQAAKCYVEANHSVPKGHCRQVVTGMAWLYFVSWGMF
PILFILGPEGLLKLSPYANSIGHSICDIIAKEFWTFLAHHLRIKIHEHILIHGDIRKTTK
EIGGEEVEVEEFVEEEDEDTV
(CsChri on; Helix 6 swap mutant including F219Y)
SEQ ID NO: 9
MSRLVAASWLLALLLCGITSTTTASSAPAASSTDGTAAAAVSHYAMNGFDELAKG
AVVPEDHFVCGPADKCYCSAWLHSRGTPGEKIGAQVCQWIAFSIAIALLTFYGFS
AWKATCGWEEVYVCCVEVLFVTLEIFKEFSSPATVYLSTGNHAYCLRYFEWLLS
CPVILIKLSNLSGLKNDYSKRTMGLIVSCVGMIVFGMAAGLATDWLKWLLYIVSCIY
GGYMYFQAAKCYVEANHSVPKGHCRQVVTGMAWLYFVSWGMFPILFILGPEGL
LKLSPYANSIGHSICDIIAKEFWTFLAHHLRIKIHEHILIHGDIRKTTKMEIGGEEVEV
EEFVEEEDEDTV

### EXAMPLE

### Example 1—Identifying Mutations which Accelerates the Off-Kinetics

The inventors' objective was to identify residues within ChR-2 whose mutations further accelerate the off-kinetics. The inventors focused on the sixth transmembrane domain.

NG108-15 cells heterologously expressing ChR2-YFP, ChR2-YFP F219Y, ReaChR-Citrine, ReaChR-Citrine F259Y, VChR1-YFP and VChR1-YFP F214Y were investigated by patch-clamp measurements in the whole cell configuration at a clamped potential of −60 mV. The bath solution contained 140 mM NaCl, 2 mM CaCl₂, 2 mM MgCl₂, 10 mM HEPES, pH 7.4 and the pipette solution contained 110 mM NaCl, 2 mM MgCl₂, 10 mM EGTA, 10 mM HEPES, pH 7.4. Photocurrents were measured in response to 500 ms light pulses with an intensity of 23 mW/mm² and a wavelength of 594 nm. The T_{off} value was determined by a fit of the current after cessation of illumination to a monoexponential function.

In order to assess the permeability of calcium ions relative to the permeability of sodium ions (P_{Ca}/P_{Na}), we measured photocurrent-voltage relationships and determined the reversal potential. The intracellular solution contained 110 mM NaCl, 10 mM EGTA, 2 mM MgCl₂ and 10 mM Tris (pH=7.4) and the extracellular solution contained 140 mM NaCl, 2 mM MgCl₂ and 10 mM Tris (pH=9). For the determination of the P_{Ca}/P_{Na} values, external 140 mM NaCl was exchanged with 90 mM CaC₂. Permeability ratios were calculated according to the Goldman-Hodgkin-Katz equation (Jan, L. Y. and Jan, Y. N. J. Physiol. 262, 215-236 (1976)).

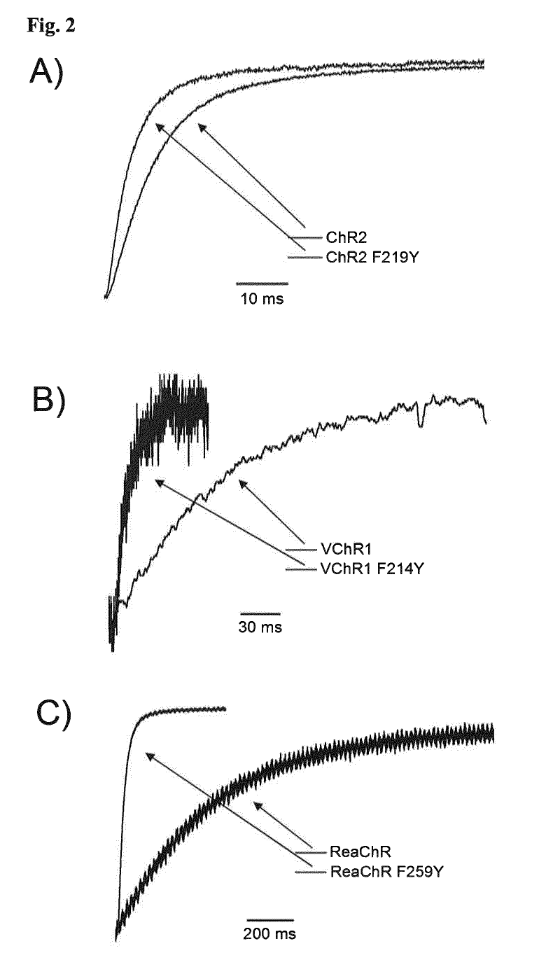

The results are shown in FIG. 2, and summarized in Table 1 below.

TABLE 1
Off-kinetics (T_{off}) and relative calcium permeabilities
(P_{Ca}/P_{Na}) of channelrhodopsin variants. Shown are the average
T_{off} values (n = 3-9), the average, relative calcium permeabilities
(n = 3-5) and the corresponding standard deviations.

| Channelrhodopsin | | |
|---|---|---|
| variant | T_{off} [ms] | P_{Ca}/P_{Na} |
| ChR2 | 9.5 ± 2.8 ms | 0.13 ± 0.01 ^{a} |
| ChR2 F219Y | 5.2 ± 1.3 ms | 0.30 ± 0.02 ^{a} |
| ReaChR | 361.0 ± 75.8 ms | 0.14 ± 0.02 ^{b} |
| ReaChR F259Y | 28.8 ± 3.8 ms | 0.22 ± 0.02 ^{b} |
| VChR1 | 119.7 ± 9.7 ms | — |
| VChR1 F214Y | 12.6 ± 1.6 ms | — |

^{a} The relative calcium permeabilites were determined in HEK293 cells.
^{b} The relative calcium permeabilities were determined in NG108-15 cells.

### Example 2—Optogenetic Stimulation of the Auditory Pathway

Hernandez et al. J Clin Invest. 2014; 124(3):1114-29, demonstrates a strategy for optogenetic stimulation of the auditory pathway in rodents. In particular, the authors describe animal models to characterize optogenetic stimulation, which is the optical stimulation of neurons genetically engineered to express the light-gated ion channel channelrhodopsin-2 (ChR2). Optogenetic stimulation of spiral ganglion neurons (SGNs) activates the auditory pathway, as demonstrated by recordings of single neuron and neuronal population responses. Furthermore, optogenetic stimulation of SGNs restore auditory activity in deaf mice. Approximation of the spatial spread of cochlear excitation by recording local field potentials (LFPs) in the inferior colliculus in response to suprathreshold optical, acoustic, and electrical stimuli indicate that optogenetic stimulation achieves better frequency resolution than monopolar electrical stimulation.

Introducing the mutations identified herein into the constructs as described, e.g., by Hernandez et al. represents routine practice. Alternatively, one may simply replace the coding sequence of the ChR2 in the constructs by the coding sequence for the light-inducible ion channel of the present disclosure.

### Example 3—Optogenetic Approach for the Recovery of Vision

Macé et al. Mol Ther. 2015; 23(1):7-16, is an earlier publication authored by some of the inventors describing optogenetic reactivation of retinal neurons mediated by adeno-associated virus (AAV) gene therapy. Most inherited retinal dystrophies display progressive photoreceptor cell degeneration leading to severe visual impairment. Optogenetic reactivation of retinal neurons mediated by adeno-associated virus (AAV) gene therapy has the potential to restore vision regardless of patient-specific mutations. The challenge for clinical translatability is to restore a vision as close to natural vision as possible, while using a surgically safe delivery route for the fragile degenerated retina. To preserve the visual processing of the inner retina, ON bipolar cells are targeted, which are still present at late stages of disease. For safe gene delivery, a recently engineered AAV variant is used that can transduce the bipolar cells after injection into the eye's easily accessible vitreous humor. It is shown that AAV encoding channelrhodopsin under the ON bipolar cell-specific promoter mediates long-term gene delivery restricted to ON-bipolar cells after intravitreal administration. Channelrhodopsin expression in ON bipolar cells leads to restoration of ON and OFF responses at the retinal and cortical levels. Moreover, light-induced locomotory behavior is restored in treated blind mice.

Introducing the mutations identified herein into the constructs as described, e.g., by Macé et al. represents routine practice. Alternatively, one may simply replace the coding sequence of the channelrhodopsins in the constructs by the coding sequence for the light-inducible ion channel of the present disclosure. The new light-inducible ion channels of the present disclosure are inserted in the cassettes for the activation of ON bipolar cells as well as for the Ganglion cells in the retina.

### LIST OF REFERENCES

U.S. Pat. No. 8,759,492 B2
WO 03/084994
WO 2012/032103
WO 2013/071231
1. Nagel, G. et al. Channelrhodopsin-2, a directly light-gated cation-selective membrane channel. Proc Natl Acad Sci USA 100, 13940-13945 (2003).
2. Nagel, G. et al. Light activation of channelrhodopsin-2 in excitable cells of Caenorhabditis elegans triggers rapid behavioral responses. Curr Biol 15, 2279-2284 (2005).
3. Boyden, E., Zhang, F., Bamberg, E., Nagel, G. & Deisseroth, K. Millisecond-timescale, genetically targeted optical control of neural activity. Nat Neurosci 8, 1263-1268 (2005).
4. Zhang, F. et al. Multimodal fast optical interrogation of neural circuitry. Nature 446, 633-639 (2007).
5. Nagel, G. et al. Channelrhodopsin-1: A Light-Gated Proton Channel in Green Algae. Science. 296, 2395-2398 (2002).
6. Bamann, C., Gueta, R., Kleinlogel, S., Nagel, G. & Bamberg, E. Structural guidance of the photocycle of channelrhodopsin-2 by an interhelical hydrogen bond. Biochemistry 49, 267-278 (2010).
7. Berndt, A., Yizhar, O., Gunaydin, L., Hegemann, P. & Deisseroth, K. Bi-stable neural state switches. Nat Neurosci 12, 229-234 (2009).
8. Lin, J., Lin, M., Steinbach, P. & Tsien, R. Characterization of engineered channelrhodopsin variants with improved properties and kinetics. Biophys J 96, 1803-1814 (2009).
9. Klapoetke N. et al. Independent optical excitation of distinct neural populations. Nature Methods. 11, 338-346 (2014).
10. Zhang F. et al. Red-shifted optogenetic excitation: a tool for fast neural control derived from Volvox carteri. Nature Neuroscience. 11, 631-633 (2008).
11. Lin J. Y. et al. ReaChR: a red-shifted variant of channelrhodopsin enables deep transcranial optogenetic excitation. Nature Neuroscience. 16, 1499-1508 (2013).
12. Kleinlogel S. et al. Ultra light-sensitive and fast neuronal activation with the Ca-permeable channelrhodopsin CatCh. Nature Neuroscience. 14, 513-518 (2011).
13. Hernandez et al. Optogenetic stimulation of the auditory pathway. J Clin Invest. 124(3): 1114-1129 (2014).
14. Macé et al. Targeting channelrhodopsin-2 to ON-bipolar cells with vitreally administered AAV Restores ON and OFF visual responses in blind mice. Mol Ther. 23(1): 7-16 (2015).
15. Jan, L. Y. and Jan, Y. N. L-Glutamate as an excitatory transmitter at the Drosophila larval neuromuscular junction. J. Physiol. 262, 215-236 (1976)
16. Kato, H. E. et al. Crystal structure of the channelrhodopsin light-gated cation channel. Nature. 428, 369-374 (2012)

1. A mutant light-inducible ion channel, wherein the mutant light-inducible ion channel comprises an amino acid sequence selected from the group consisting of A, B and a combination thereof:
A: an amino acid sequence which has at least 84% similarity to the full length sequence of SEQ ID NO: 1 (ChR-2);
B: an amino acid sequence which has at least 75% identity to the full length sequence of SEQ ID NO: 1 (ChR-2);
and wherein the mutant light-inducible ion channel only differs from its parent light-inducible ion channel by a substitution at a position corresponding to F219 in SEQ ID NO: 1,
which substitution accelerates the off-kinetics of the mutant channel as compared to the parent channel, when compared by patch-clamp measurements in the whole cell configuration at a clamp potential of −60 mV, a bath solution of 140 mM NaCl, 2 mM CaCl₂), 2 MgCl₂, 10 mM HEPES, pH 7.4, and a pipette solution of 110 mM NaCl, 2 mM MgCl₂, 10 mM EGTA, 10 mM HEPES, pH 7.4.

2. The mutant light-inducible ion channel of claim 1, wherein the mutant light-inducible ion channel is selected from C, D and a combination thereof:
C: a mutant light-inducible ion channel which has at least 86% similarity to the full length of SEQ ID NO: 1 (ChR-2);
D: a mutant light-inducible ion channel which has at least 80% identity to the full length of SEQ ID NO: 1 (ChR-2).

3. The mutant light-inducible ion channel of claim 1, wherein the substitution is F219Y.

4. The mutant light-inducible ion channel of claim 3, wherein the mutant channel comprises the motif of SEQ ID NO: 4:
Cys-Arg-Xaa₃-Xaa₄-Val-Xaa₆-Xaa₇-Met-Ala-Trp-Xaa₁₁-
Tyr-Phe-Val-Xaa₁₅-Trp-Gly-Met-Phe-Pro-Xaa₂₁-Leu-Phe-
Xaa₂₄-Leu,
wherein Xaa₃ is Gin or Glu;
wherein Xaa₄ is Val or Leu;
wherein Xaa₆ is Thr or Arg;
wherein Xaa₇ is Gly, Val or Ala;
wherein Xaa₁₁ is Leu or Thr;
wherein Xaa₁₅ is Ser or Ala;
wherein Xaa₂₁ is Ile or Val; and
wherein Xaa₂₄ is Ile or Leu.

5. The mutant light-inducible ion channel of claim 1, which mutant channel further comprises Cys, Ser, Glu, Asp, or Thr at a position corresponding to L132 in SEQ ID NO: 1.

6. The mutant light-inducible ion channel of claim 1, wherein
the mutant light-inducible ion channel comprises the amino acid sequence of SEQ ID NO: 1 (ChR-2), except for said substitution at position F219, and optionally the amino acid at position 132 of SEQ ID NO: 1; or
wherein the mutant light-inducible ion channel comprises the amino acid sequence of SEQ ID NO: 2 (VChR1), except for said substitution at position F214, and optionally the amino acid at the position in SEQ ID NO: 2 corresponding to L132 in SEQ ID NO: 1; or
wherein the mutant light-inducible ion channel comprises the amino acid sequence of SEQ ID NO: 3 (ReaChR), except for said substitution at position F259, and optionally the amino acid at the position in SEQ ID NO: 3 corresponding to L132 in SEQ ID NO: 1.

7. The mutant light-inducible ion channel of claim 1, wherein the light-inducible ion channel additionally comprises one or more of the following amino acid residues: aspartic acid at a position corresponding to position 253 of SEQ ID NO: 1; lysine at a position corresponding to position 257 of SEQ ID NO: 1; tryptophan at a position corresponding to position 260 of SEQ ID NO: 1; glutamic acid at a position corresponding to position 123 of SEQ ID NO: 1; histidine or arginine, at a position corresponding to position 134 of SEQ ID NO: 1; threonine, serine, or alanine at a position corresponding to position 128 of SEQ ID NO: 1; and alanine at a position corresponding to position 156 of SEQ ID NO: 1.

8. A nucleic acid construct, comprising a nucleotide sequence coding for the mutant light-inducible ion channel according to claim 1.

9. An expression vector, comprising a nucleotide sequence coding for the light-inducible ion channel according to claim 1.

10. A cell comprising the nucleic acid construct according to claim 8.

11. The cell of claim 10, wherein the cell is a mammalian cell.

12. Hiqh-throuqhput screening method of using a light-inducible ion channel according to claim 1 comprising the step of providing said light-inducible ion channel or said cell.

13. Method for light-stimulation of neuron cells comprising applying a light stimulus to a cell comprising a light-inducible ion channel according to claim 1.

14. (canceled)

15. A non-human animal, comprising a light-inducible ion channel according to claim 1.

16. The mutant light-inducible ion channel of claim 4, wherein the mutant channel comprises the motif of SEQ ID NO: 4:
Cys-Arg-Xaa₃-Xaa₄-Val-Xaa₆-Xaa₇-Met-Ala-Trp-Xaa₁₁-
Tyr-Phe-Val-Xaa₁₅-Trp-Gly-Met-Phe-Pro-Xaa₂₁-Leu-Phe-
Xaa₂₄-Leu,
wherein Xaa₃ is Gln;
wherein Xaa₄ is Val;
wherein Xaa₆ is Thr;
wherein Xaa₇ is Gly;
wherein Xaa₁₁ is Leu;
wherein Xaa₁₅ is Ser;
wherein Xaa₂₁ is Ile; and
wherein Xaa₂₄ is Ile.

17. The mutant light-inducible ion channel of claim 5, wherein the mutant channel comprises Cys at a position corresponding to L132 in SEQ ID NO: 1.

18. The cell of claim 11, wherein the cell is selected from the group consisting of
(a) a hippocampal cell, a photoreceptor cell, a retinal rod cell, a retinal cone cell, a retinal ganglion cell, a bipolar neuron, a ganglion cell, a pseudounipolar neuron, a multipolar neuron, a pyramidal neuron, a Purkinje cell, or a granule cell; and
(b) a neuroblastoma cell; a HEK293 cell; a COS cell; a BHK cell; a CHO cell; a myeloma cell; or a MDCK cell.

19. The cell of claim 10, wherein the cell is a NG108-15 neuroblastoma cell.


SEQUENCE LISTING (9 sequences)

SEQ ID NO: 1 - protein, 315 aa, Chlamydomonas reinhardtii
MDYGGALSAVGRELLFVTNPVVVNGSVLVPEDQCYCAGWIESRGTNGAQTASNVLQWLAAGFSILLLMFYAYQTWKSTCGWEEIYVCAIEMVKVILEFFFEFKNPSMLYLATGHRVQWLRYAEWLLTCPVILIHLSNLTGLSNDYSRRTMGLLVSDIGTIVWGATSAMATGYVKVIFFCLGLCYGANTFFHAAKAYIEGYHTVPKGRCRQVVTGMAWLFFVSWGMFPILFILGPEGFGVLSVYGSTVGHTIIDLMSKNCWGLLGHYLRVLIHEHILIHGDIRKTTKLNIGGTEIEVETLVEDEAEAGAVNKGTGK

SEQ ID NO: 2 - protein, 300 aa, Volvox carteri
MDYPVARSLIVRYPTDLGNGTVCMPRGQCYCEGWLRSRGTSIEKTIAITLQWVVFALSVACLGWYAYQAWRATCGWEEVYVALIEMMKSIIEAFHEFDSPATLWLSSGNGVVWMRYGEWLLTCPVLLIHLSNLTGLKDDYSKRTMGLLVSDVGCIVWGATSAMCTGWTKILFFLISLSYGMYTYFHAAKVYIEAFHTVPKGICRELVRVMAWTFFVAWGMFPVLFLLGTEGFGHISPYGSAIGHSILDLIAKNMWGVLGNYLRVKIHEHILLYGDIRKKQKITIAGQEMEVETLVAEEED

SEQ ID NO: 3 - protein, 352 aa, Artificial
  ReaChR
MVSRRPWLLALALAVALAAGSAGASTGSDATVPVATQDGPDYVFHRAHERMLFQTSYTLENNGSVICIPNNGQCFCLAWLKSNGTNAEKLAANILQWVVFALSVACLGWYAYQAWRATCGWEEVYVALIEMMKSIIEAFHEFDSPATLWLSSGNGVVWMRYGEWLLTCPVILIHLSNLTGLKDDYSKRTMGLLVSDVGCIVWGATSAMCTGWTKILFFLISLSYGMYTYFHAAKVYIEAFHTVPKGLCRQLVRAMAWLFFVSWGMFPVLFLLGPEGFGHISPYGSAIGHSILDLIAKNMWGVLGNYLRVKIHEHILLYGDIRKKQKITIAGQEMEVETLVAEEEDKYESSLE

SEQ ID NO: 4 - protein, 25 aa, Artificial
  Helix 6 consensus motif
  VARIANT (3)..(3): wherein Xaa3 is Gln or Glu, in particular wherein Xaa3 is Gln
  VARIANT (4)..(4): wherein Xaa4 is Val or Leu, in particular wherein Xaa4 is Val
  VARIANT (6)..(6): wherein Xaa6 is Thr or Arg, in particular wherein Xaa6 is Thr
  VARIANT (7)..(7): wherein Xaa7 is Gly, Val or Ala, in particular wherein Xaa7 is Gly
  VARIANT (11)..(11): wherein Xaa11 is Leu or Thr, in particular wherein Xaa11 is Leu
  VARIANT (15)..(15): wherein Xaa15 is Ser or Ala, in particular wherein Xaa15 is Ser
  VARIANT (21)..(21): wherein Xaa21 is Ile or Val, in particular wherein Xaa21 is Ile
  VARIANT (24)..(24): wherein Xaa24 is Ile or Leu, in particular wherein Xaa24 is Ile
CRXXVXXMAWXYFVXWGMFPXLFXL

SEQ ID NO: 5 - protein, 9 aa, Artificial
  Retinal binding site consensus motif
  VARIANT (1)..(1): wherein Xaa1 is Leu, Ile, Ala or Cys
  VARIANT (3)..(5): wherein Xaa3, Xaa4, and Xaa5 is independently any amino acid
  VARIANT (7)..(8): wherein Xaa7 and Xaa8 is independently any amino acid
  VARIANT (9)..(9): wherein Xaa9 is Thr, Phe, or Tyr
XDXXXKXXX

SEQ ID NO: 6 - protein, 349 aa, Artificial
  Chrimson derived from Chlamydomonas noctigama
MAELISSATRSLFAAGGINPWPNPYHHEDMGCGGMTPTGECFSTEWWCDPSYGLSDAGYGYCFVEATGGYLVVGVEKKQAWLHSRGTPGEKIGAQVCQWIAFSIAIALLTFYGFSAWKATCGWEEVYVCCVEVLFVTLEIFKEFSSPATVYLSTGNHAYCLRYFEWLLSCPVILIKLSNLSGLKNDYSKRTMGLIVSCVGMIVFGMAAGLATDWLKWLLYIVSCIYGGYMYFQAAKCYVEANHSVPKGHCRMVVKLMAYAYFASWGSYPILWAVGPEGLLKLSPYANSIGHSICDIIAKEFWTFLAHHLRIKIHEHILIHGDIRKTTKEIGGEEVEVEEFVEEEDEDTV

SEQ ID NO: 7 - protein, 345 aa, Artificial
  CSChrimson
MSRLVAASWLLALLLCGITSTTTASSAPAASSTDGTAAAAVSHYAMNGFDELAKGAVVPEDHFVCGPADKCYCSAWLHSRGTPGEKIGAQVCQWIAFSIAIALLTFYGFSAWKATCGWEEVYVCCVEVLFVTLEIFKEFSSPATVYLSTGNHAYCLRYFEWLLSCPVILIKLSNLSGLKNDYSKRTMGLIVSCVGMIVFGMAAGLATDWLKWLLYIVSCIYGGYMYFQAAKCYVEANHSVPKGHCRMVVKLMAYAYFASWGSYPILWAVGPEGLLKLSPYANSIGHSICDIIAKEFWTFLAHHLRIKIHEHILIHGDIRKTTKMEIGGEEVEVEEFVEEEDEDTV

SEQ ID NO: 8 - protein, 348 aa, Artificial
  Chrimson Helix 6 Swap
MAELISSATRSLFAAGGINPWPNPYHHEDMGCGGMTPTGECFSTEWWCDPSYGLSDAGYGYCFVEATGGYLVVGVEKKQAWLHSRGTPGEKIGAQVCQWIAFSIAIALLTFYGFSAWKATCGWEEVYVCCVEVLFVTLEIFKEFSSPATVYLSTGNHAYCLRYFEWLLSCPVILIKLSNLSGLKNDYSKRTMGLIVSCVGMIVFGMAAGLATDWLKWLLYIVSCIYGGYMYFQAAKCYVEANHSVPKGHCRQVVTGMAWLYFVSWGMFPILFILPEGLLKLSPYANSIGHSICDIIAKEFWTFLAHHLRIKIHEHILIHGDIRKTTKEIGGEEVEVEEFVEEEDEDTV

SEQ ID NO: 9 - protein, 344 aa, Artificial
  CSChrimson Helix 6 SWAP
MSRLVAASWLLALLLCGITSTTTASSAPAASSTDGTAAAAVSHYAMNGFDELAKGAVVPEDHFVCGPADKCYCSAWLHSRGTPGEKIGAQVCQWIAFSIAIALLTFYGFSAWKATCGWEEVYVCCVEVLFVTLEIFKEFSSPATVYLSTGNHAYCLRYFEWLLSCPVILIKLSNLSGLKNDYSKRTMGLIVSCVGMIVFGMAAGLATDWLKWLLYIVSCIYGGYMYFQAAKCYVEANHSVPKGHCRQVVTGMAWLYFVSWGMFPILFILPEGLLKLSPYANSIGHSICDIIAKEFWTFLAHHLRIKIHEHILIHGDIRKTTKMEIGGEEVEVEEFVEEEDEDTV